(19) United States
(12) Patent Application Publication
(10) Pub. No.: US 2011/0082391 A1
(43) Pub. Date: Apr. 7, 2011

(54) METHODS FOR MEASURING CHANGE IN LIP SIZE AFTER AUGMENTATION

(75) Inventors: Michael A.C. Kane, Scottsdale, AZ (US); Xiaoming Lin, Scottsdale, AZ (US); Mitchell S. Wortzman, Scottsdale, AZ (US); Stacy Smith, Scottsdale, AZ (US); Zbigniew Paul Lorenc, Scottsdale, AZ (US)

(73) Assignee: MEDICIS PHARMACEUTICAL CORPORATION

(21) Appl. No.: 12/797,710

(22) Filed: Jun. 10, 2010

Related U.S. Application Data
(60) Provisional application No. 61/291,213, filed on Dec. 30, 2009.
(60) Provisional application No. 61/268,411, filed on Jun. 12, 2009.

(51) Int. Cl. A61B 5/103

(52) U.S. Cl. 600/587

(57) ABSTRACT

A method for measuring the effect of a medical treatment on the size of lips. The method has the following steps: (a) providing a scale of at least four visual reference images exhibiting varying lip sizes and assigning a unique indicator to each of the at least four visual reference images; (b) visually examining a lip of a human subject to be augmented and selecting one from among of at least four different reference images most closely corresponding in lip size and identifying the corresponding unique indicator; (c) introducing into the lip of the human subject a filler or an implant to augment the size of the lip; (d) visually examining the lip after introduction of the filler or the implant and selecting one of the at least four different reference images most closely corresponding in lip size and identifying the corresponding unique indicator; and (e) comparing the unique indicator of the lip before introduction of the filler or the implant and the unique indicator of the lip after introduction of the filler or the implant to determine if they are different. There is also a method for counseling a human subject undertaking augmentation of lips. There is also a method for developing a scale for measuring differences in lip size in human subjects. There is also a method for determining the amount of filler or implant needed to augment the lips of a human subject.

### CROSS-REFERENCE TO A RELATED APPLICATION

The present application claims priority based on U.S. Provisional Application No. 61/291,213, filed Dec. 30, 2009, and U.S. Provisional Application No. 61/268,411, filed Jun. 12, 2009, both of which are incorporated herein by reference.

### BACKGROUND OF THE INVENTION

1. Field of the Invention

The present invention relates to a method for measuring change in lip size after augmentation. The present invention also relates to a method for measuring the effect of a medical treatment on the size of lips. The present invention further relates to a method for counseling a human subject undertaking augmentation of lips. The present invention still further relates to a method for developing a scale for measuring differences in lip size in human subjects. The present invention still further relates to a method for counseling a human subject undertaking augmentation of lips. The present invention still further relates to a method for developing a scale for measuring differences in lip size in human subjects. The present invention still further relates to a method for determining the amount of filler needed to augment the lips of a human subject.

2. Description of the Related Art

Lip augmentation is a cosmetic procedure undertaken to achieve fuller lips. Augmentation is normally accomplished by introducing fillers or implants into the lips. Examples of fillers are non-animal stabilized hyaluronic acid (NASHA) gels, liquid silicones, alloderm, and collagen. Fillers are typically injected. Examples of permanent implants are fats, silicone solids, and gore-tex. Implants are usually inserted surgically.

There is a need for effective tools by which physicians can communicate augmentation treatment goals to patients and measure the effect of the lip augmentation.

### SUMMARY OF THE INVENTION

According to the present invention, there is provided a method for measuring the effect of a medical treatment on the size of lips. The method has the following steps: (a) developing a scale of at least four reference images exhibiting varying lip sizes and assigning a unique indicator to each of the at least four reference images; (b) examining a lip of a human subject to be treated and selecting one of the at least four different reference images most closely corresponding in lip size and identifying the corresponding unique indicator; (c) introducing into the lip of the human subject a filler or an implant to augment the size of the lip; (d) examining the treated lip and selecting one of the at least four different reference images most closely corresponding in lip size and identifying the corresponding unique indicator; and (e) comparing the unique indicator of the lip before injection and the unique indicator of the lip after injection to determine if they are different.

Further according to the present invention, there is provided a method for counseling a human subject undertaking augmentation of lips. The method has the following steps: (a) visually examining a lip of the human subject and comparing it to a scale of at least four visual reference images exhibiting human lips of varying sizes; (b) selecting a first reference image from among the at least four different reference images that corresponds most closely in lip size to that of the human subject wherein the first reference image does not exhibit the largest lip size among the at least four different reference images; (c) selecting a second reference image from among the at least four different reference images that exhibits lips of larger size than that of first reference image; and (d) allowing the human subject to visually compare the lip size exhibited in first reference image with the lip size exhibited in second reference image.

Further according to the present invention, there is provided a method for developing a scale for measuring differences in lip size in human subjects. The method has the steps of (a) developing a scale of at least four visual reference images exhibiting varying lip sizes and having unique indicators assigned thereto; (b) subjecting the scale to a panel test of a plural number of human subjects and a plural number of evaluators who each visually examine a lip of the plural number of human subjects and assign a unique indicator to each lip; and (c) approving the scale as viable for use in human subjects if the weighted kappa coefficient for each of the unique indicators is from 0.40 to 1.0 with an associated 95% confidence interval.

Further according to the present invention, there is provided a method for determining the amount of filler needed to augment the lips of a human subject. The method has the following steps: (a) examining a lip of the human subject and comparing it to a scale of at least four reference images exhibiting human lips of varying sizes; (b) selecting a first reference image from among the at least four different reference images that corresponds most closely in lip size to that of the human subject wherein the first reference image does not exhibit the largest lip size among the at least four different reference images; (c) selecting a second reference image from among the at least four different reference images (from the remaining reference images) that exhibits lips of larger size than that of first reference image and that substantially corresponds to an augmented lip size desired by the human subject; and (d) ascertaining the amount of filler needed on the basis of a predetermined relative amount relationship between the first reference image and the second reference image.

### DESCRIPTION OF THE FIGURES

FIG. 1 is a photographic image of a lip scale for a very thin size upper lip useful in the method of the present invention.

FIG. 2 is a photographic image of a lip scale for a thin size upper lip useful in the method of the present invention.

FIG. 3 is a photographic image of a lip scale for a medium size upper lip useful in the method of the present invention.

FIG. 4 is a photographic image of a lip scale for a full size upper lip useful in the method of the present invention.

FIG. 5 is a photographic image of a lip scale for a very full size upper lip useful in the method of the present invention.

FIG. 6 is a photographic image of a lip scale for a very thin size lower lip useful in the method of the present invention.

FIG. 7 is a photographic image of a lip scale for a thin size lower lip useful in the method of the present invention.

FIG. 8 is a photographic image of a lip scale for a medium size lower lip useful in the method of the present invention.

FIG. 9 is a photographic image of a lip scale for a full size lower lip useful in the method of the present invention.

FIG. 10 is a photographic image of a lip scale for a very full size lower lip useful in the method of the present invention.

### DETAILED DESCRIPTION OF THE INVENTION

The present invention provides tools by which physicians can communicate and discuss treatment goals with patients as well as to measure the treatment effect of the lip augmentation, the extent of augmentation in the lips can be measured by the use of sets of lip scales.

The present invention in a preferred embodiment provides a set of lip scales for the upper lip and another set of lip scales for the lower lip. If desired, a set of lip scales can be provided with the upper and lower lips together.

Each set takes the form of at least four reference images. Preferred sets take the form of four to six reference images. Most preferred sets take the form of five reference images. The number of reference images is selected so that there are enough to encompass and depict normal variation in lip size yet not so many as to render difficult the differences in size progression of images by an analyzing computer or normal human visual identification.

Reference images can take the form of any known in the art to convey the shape and size of the lips with clarity sufficient for normal human visual identification. For example, reference images may take the form of drawings or live photographs. Reference images may also take the form of computer-generated images. Photographs are preferred for human visual identification as they can more effectively depict the effects of ageing. Reference images may be in black-and-white or in color. Colored reference images are preferred.

Reference images are assigned unique indicators for the purpose of identification. For example, unique numerals, letters, words, or combinations thereof are possible. For simplicity and for ease of mathematical manipulation and analysis, numerals are preferred. In the embodiment disclosed herein, the unique numerals 1 to 5 have been selected.

The lips of a human subject to be augmented are examined visually both before and after augmentation to detect differences in size. The reference image most closely corresponding in lip size to that of the human subject before augmentation is selected and a unique indicator is identified. The image most closely corresponding in lip size to that of the human subject after augmentation is also selected and a unique indicator is identified. In a desirable scenario, the images before and after augmentation will be different so as to indicate an increase in size of the lips after augmentation. Visual examination can be carried out by a person with normal or better eyesight, e.g., about 20:20 (corrected or uncorrected).

Lips are typically augmented by introduction of fillers or implants into the lips. Examples of fillers are non-animal stabilized hyaluronic acid (NASHA) gels, collagen, liquid silicones, poly-L-lactic acid (PLA), and alloderm. NASHA gels are preferred and are available commercially as Restylane® by Medicis Pharmaceutical Corp. Injectable PLAs are available commercially as Sculptra® by Sanofi-Aventis. Another useful filler is calcium hydroxylaptite (CaHA) microspheres suspended in a sodium carboxymethylcellose gel, such as Radiesse® by Bio-Form Inc. Fillers are typically introduced into the lips by injection via syringe. Examples of materials suitable for permanent implants are fats, silicone solids, and gore-tex.

Lip size is characterized generally on the basis of the relative volume (two or three dimensional) of the upper and/or lower lips without reference to any particular linear dimension as being controlling. Linear dimensions and/or lip areas that can impact lip size or lip volume include, but are not limited to, total vermilion height, upper red lip median height, upper red lip lateral height, and lower lip median height, upper lip vermilion area, lower lip vermilion area, and combinations of the foregoing. If desired, lip size or volume can be characterized as total lip volume (upper and lower combined).

In addition to larger lip size, augmentation via introduction of a filler or an implant can afford more youthful-looking lips and can provide more definition of anatomical landmarks, such as a cupid's bow and philtral columns.

The lip scales are also useful to physicians in communicating treatment goals to patients and providing counseling regarding same. For instance, a patient of a particular size lip could be counseled that an augmentation procedure is anticipated to result in larger lips commensurate in size with a particular visual reference image or images.

Another aspect of the invention is when the lip scales are used as an aid in counseling patients. A feature of the invention is selection of a first image by the physician from among four or more images of lips of varying sizes wherein the first image corresponds most closely in size to that of the patient. A second image of larger size is then selected by the physician as a visual aid for the benefit of the patient to compare with the first image. The second image could be the next size larger than the first image or could be two or more sizes larger. The second image can be used to demonstrate what larger lips would look like and can be used by the patient to convey to the physician how full they want their lips to be.

The method for counseling a human subject undertaking augmentation of lips has the following steps: (a) visually examining a lip of the human subject and comparing it to a scale of at least four visual reference images exhibiting human lips of varying sizes; (b) selecting a first reference image from among the at least four different reference images that corresponds most closely in lip size to that of the human subject wherein the first reference image does not exhibit the largest lip size among the at least four different reference images; (c) selecting a second reference image from among the at least four different reference images that exhibits lips of larger size than that of first reference image; and (d) allowing the human subject to visually compare the lip size exhibited in first reference image with the lip size exhibited in second reference image.

Another aspect of the invention is a method for developing a scale for measuring differences in lip size in human subjects. The method has the steps of (a) developing a scale of at least four visual reference images exhibiting varying lip sizes and having unique indicators assigned thereto; (b) subjecting the scale to a panel test of a plural number of human subjects and a plural number of evaluators who each visually examine a lip of the plural number of human subjects and assign a unique indicator to each lip; and (c) approving the scale as viable for use in human subjects if the weighted kappa coefficient for each of the unique indicators is from 0.40 to 1.0 with an associated 95% confidence interval.

The panel test in scale development results in at least four visual reference images, preferably from four to six images, and most preferably five images.

The panel test in scale development utilizes a plural number of human subjects and a plural number of evaluators, both in statistically sufficient number to provide the indicated weighted kappa coefficient of 0.40 to 1.0 with an associated 95% confidence interval for the unique indicators. A preferred weighted kappa coefficient is about 0.60 to 1.0. A most preferred weighted kappa coefficient is about 0.80 to 1.0.

The number of human subjects in the panel tests preferably ranges from about 25 to about 150 subjects, more preferably from about 50 to about 100 subjects, and most preferably about 75 to about 85 subjects. The number of evaluators in the panel test preferably ranges from about 2 to about 12 evaluators, more preferably about 3 to about 10 evaluators, and most preferably about 4 to about 6 evaluators.

Human subjects can be selected from either or both of the sexes or from any race or combinations of races. Preferably, reference visual images are selected in size and number such that a set of lip scales is applicable to any race or all races. Examples of races useful as subjects for reference visual images include, but are not limited to, Caucasian (generally white), Negro (generally black), and Oriental. Humans of mixed race and of races not amenable to ready categorization are also useful as subjects for reference visual images.

Another aspect of the invention is that it can be used as a tool for determining the amount of filler needed to augment the lips of a human subject. The relative lip size variation between different reference images can be correlated to particular amounts of filler necessary to augment the lips to larger sizes. A method for determining the amount of filler has the following steps: (a) visually examining a lip of the human subject and comparing it to a scale of at least four visual reference images exhibiting human lips of varying sizes; (b) selecting a first reference image from among the at least four different reference images that corresponds most closely in lip size to that of the human subject wherein the first reference image does not exhibit the largest lip size among the at least four different reference images; (c) selecting a second reference image from among the at least four different reference images (from the remaining reference images) that exhibits lips of larger size than that of first reference image and that substantially corresponds to an augmented lip size desired by the human subject; and (d) ascertaining the amount of filler needed on the basis of a predetermined relative amount relationship between the first reference image and the second reference image.

The following are examples of the present invention and are not to be construed as limiting.

### EXAMPLES

A photographic grading system for evaluating the effects of augmentation of lip soft tissue volume was undertaken. The 5-point photographic scale is used to grade lip fullness ranging in severity from Very Thin (Grade 1) to Very Full (Grade 5) for each lip (upper and lower) separately.

The photographic grading system, also referred to as the 5-point Lip Fullness Scales (LFS), was validated for the purpose of demonstrating its accuracy. Provided are the background on the development of the LFS, the method of selection of photos for the LFS, the method used to validate the LFS, and the results of the validation.

### Objectives

An objective was to evaluate the 5-graded Lip Fullness Scales (LFS) regarding the within-evaluator and between-evaluator agreement. There were two separate Lip Fullness Scales, one for the upper lip and one for the lower lip. The within-observer agreement refers to the ability of each evaluator to reproduce their original score at a subsequent time, having allowed reasonable amount of time to elapse so that memory was not a likely factor. Between-observer agreement is the degree to which the evaluators independently provided the same score for the same subject.

### Validation Procedure

The validation study included 85 photographs that were assessed independently by five board-certified dermatologists or plastic surgeons (Evaluators). Photographs were chosen for upper lips and lower lips, separately; 76 of the 85 chosen were used for both the upper and lower lip scale validation. Each photograph displayed a frontal (AP) view of the lips slightly parted. The Evaluators rated the lip fullness using the 5-graded LFS described below. The photographs used aimed to reflect the range of the scale, ratings 1 to 5. Each photograph had a unique identification number, but they were not arranged in any specific order.

Assessments were made by each of the Evaluators at two occasions, at least 2 weeks apart. The same set of photographs was used for both occasions, but the photographs were provided to the Evaluators in a different order at each time.

Each score in the LFS was exemplified by a set of at least three photographs. None of the photographs by which the scale was exemplified were used in the sets of photographs tested. The exemplifying photographs were selected by the Evaluators before the validation was performed. The LFS is presented in Table 1.

TABLE 1
(Lip Fullness Scales)

| | | | Lip Fullness Scales |
|---|---|---|---|
| Grade | Lip Fullness Scales (Upper) | Grade | (Lower) |
| 1 | Very Thin | 1 | Very Thin |
| 2 | Thin | 2 | Thin |
| 3 | Medium | 3 | Medium |
| 4 | Full | 4 | Full |
| 5 | Very Full | 5 | Very Full |

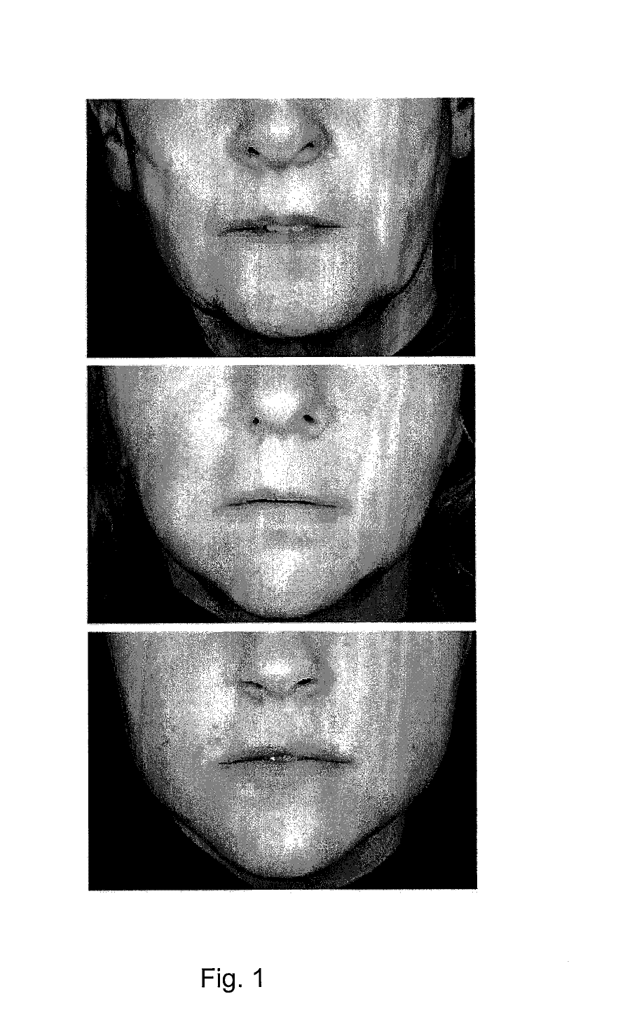

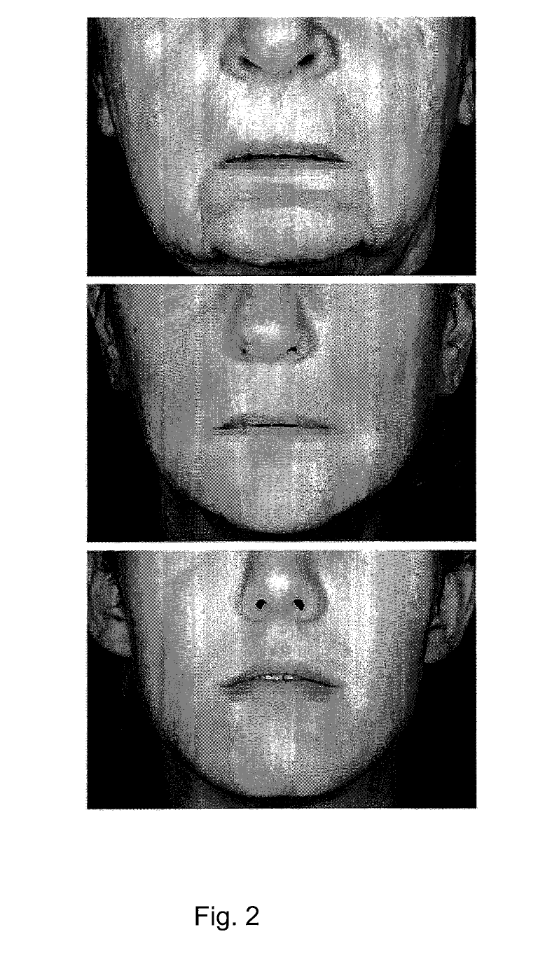

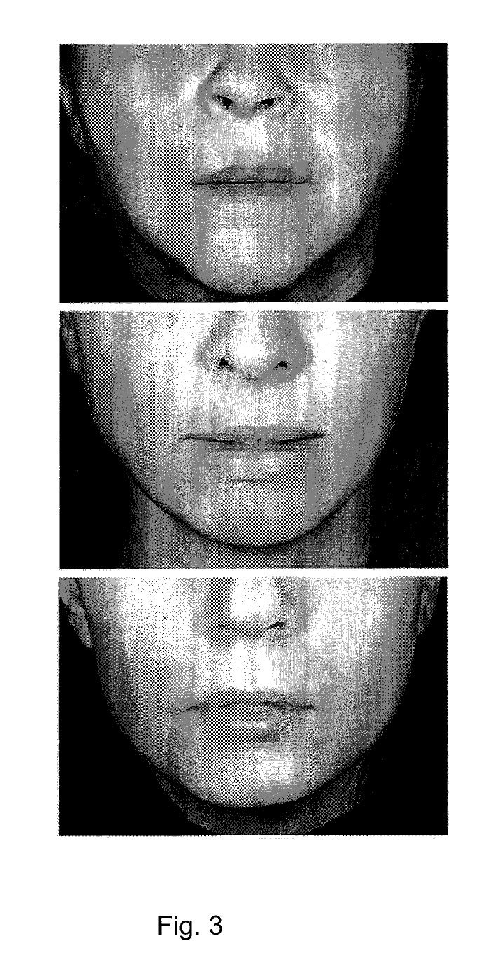

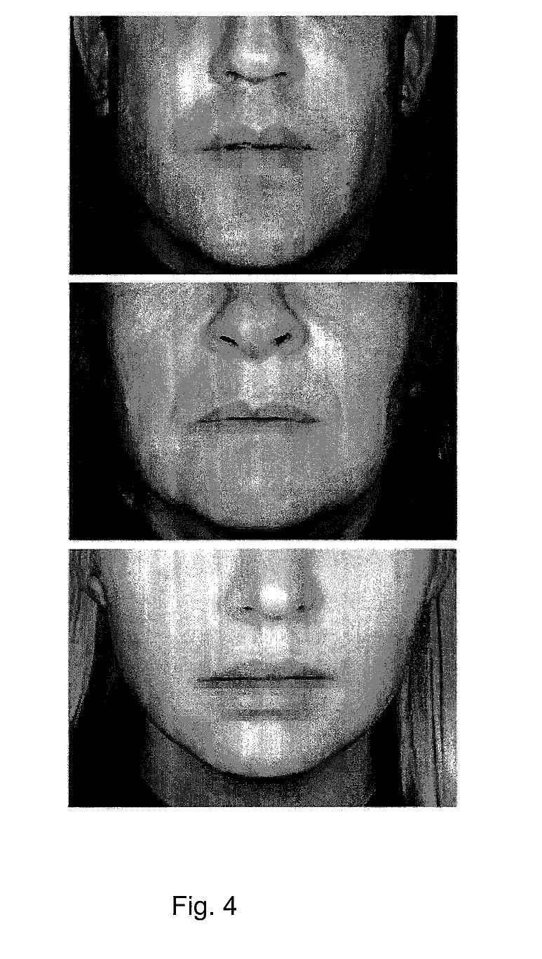

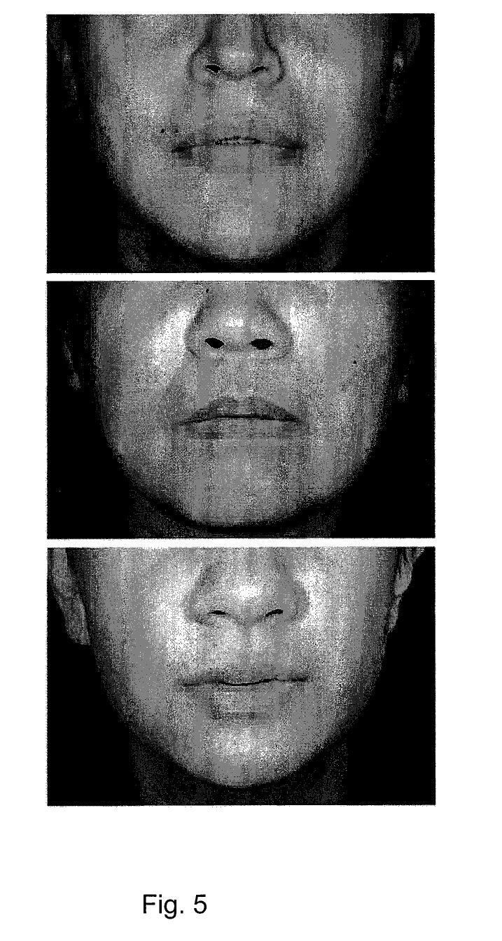

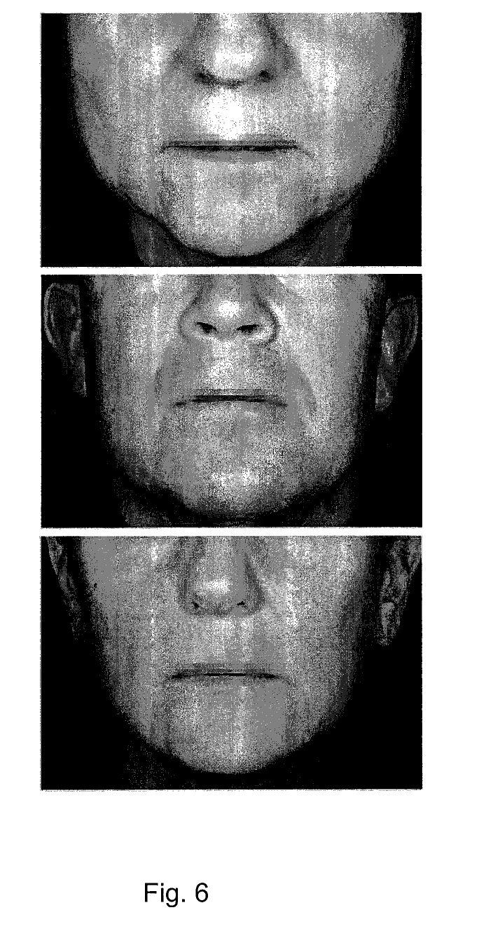

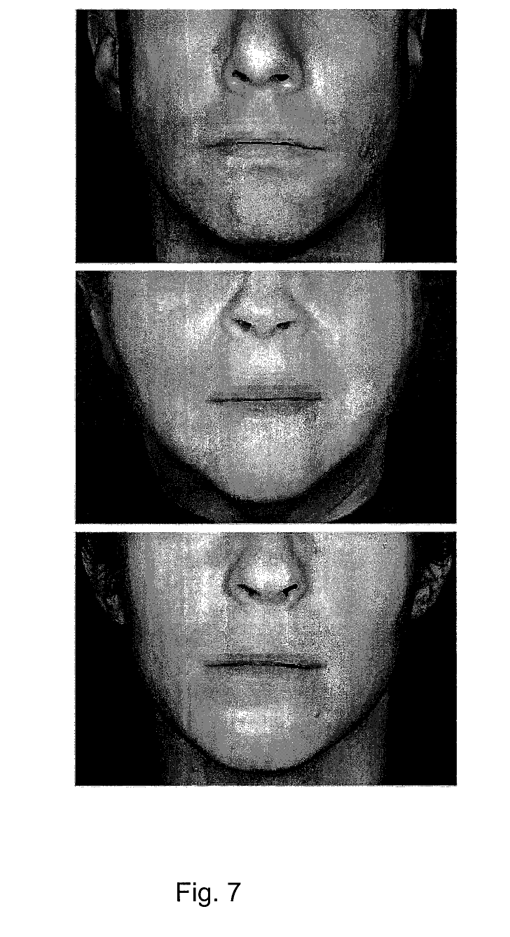

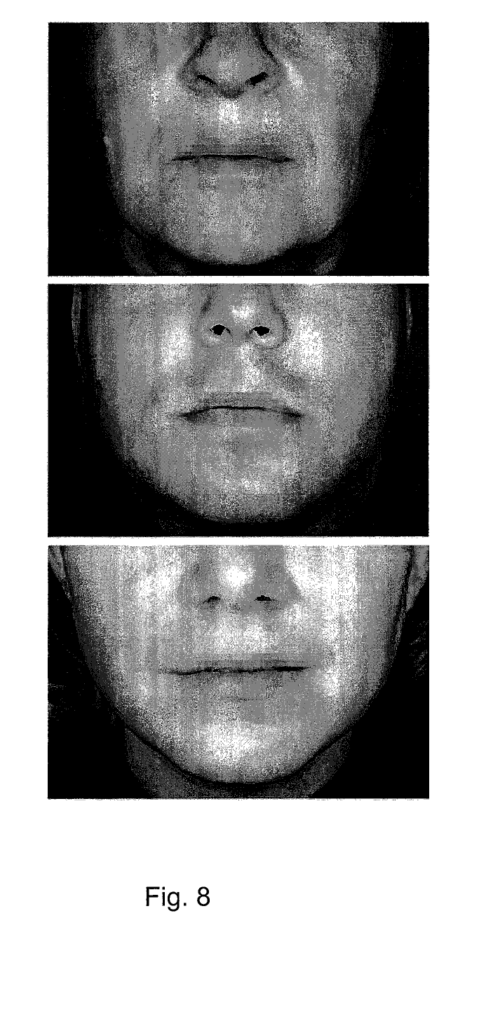

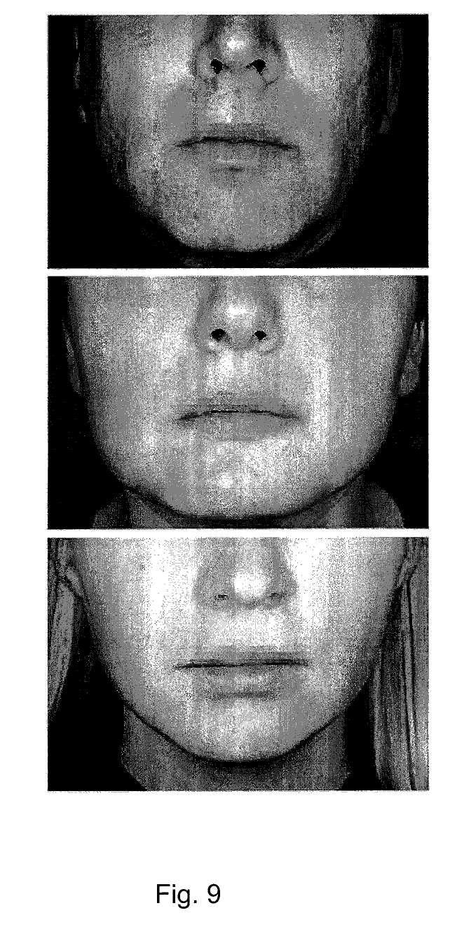

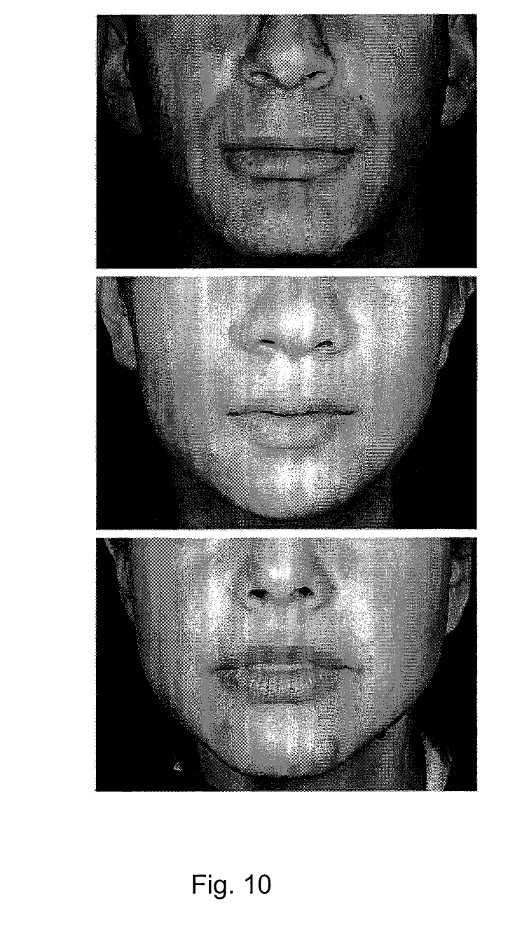

Each Evaluator received the Lip Fullness Scales, including exemplifying photographs set forth in FIGS. 1 to 10 and the set of photographs to be tested. The assessments were made individually and the results recorded in validation review booklets. Assessments were not discussed between the Evaluators.

### Randomization

A photo list was randomized using a standardized hypertext preprocessor (php) based computer randomization program. Each photo was randomly assigned to a sequence number. This randomization was conducted twice in order to create two separate randomization lists.

### Statistical Methods

### Within-Observer Agreement:

Five evaluators evaluated each of the 85 photographs at two occasions. The agreement of these matched data was assessed using two measures utilizing the original data on the 5-graded LFS (separately for the upper and lower lip): (1) the overall proportion of the observed agreement, i.e. the sum of the number of ratings in the main diagonal of the square matrix, divided by the total number of observations and (2) a weighted kappa coefficient and associated 95% confidence interval. A value of the weighted kappa coefficient ≧0.75 is considered as excellent agreement, whereas a value ≦0.40 signifies poor agreement.

### Between-Observer Agreement:

The overall proportion of the observed agreement, i.e. the sum of the number of ratings in the main diagonal of the square matrix, divided by the total number of evaluations was calculated for the ten pairs of evaluators, separately for the upper and lower lip scales.

Pair-wise weighted kappa coefficients were calculated (along with associated 95% confidence intervals) for the five Evaluators, resulting in ten weighted kappa coefficients for each scale. In addition, an overall kappa value based on all 5 Evaluators was generated for each scale. A value of the weighted kappa coefficient ≧0.75 is considered as excellent agreement, whereas a value ≧0.40 signifies poor agreement.

### Determination of Sample Size

Sample size was chosen based on logistical considerations. However, with five Evaluators each assessing 80 photographs, the weighted kappa coefficient can be calculated within 0.084 points (assuming 60% agreement and 95% confidence level).

### Changes in the Conduct of the Evaluation or Planned Analyses

Not all of the same photos were used for the upper and lower lip in order to have better presentation of the full spectrum of lip fullness. The within-observer weighted kappa values were stratified by rater and the between-observer weighted kappa values were stratified by round of review. The interpretation of kappa values was modified to reflect the current literature. A validation was added that compared intra-rater live vs. photographic assessment.

Results from Photographic Validation

### Study Subjects:

The first validation study included no live patients. Photographs were used for all assessments.

Table 2 summarizes the demographic characteristics of the subjects used to photographically exemplify the upper and lower lips for this validation. A total of 85 subject photographs were used to illustrate the upper lip and 85 subject photographs were used to illustrate the upper lip; 76 of the 85 cases used the same photographs and in 9 cases the upper and lower lip used different photographs.

The mean age of both the upper and lower lip groups of subjects was 40 years, with the age range of 18 to 76 years for the upper lip and 18 to 75 years for the lower lip. Approximately half the subjects in both groups were 18 to 34 years of age. The majority of subjects in both groups were of female (62% for the upper lip and 66% for the lower lip) and Caucasian (84% and 80% for upper and lower lip), respectively. Both groups were composed of 5% African Americans (blacks). Hispanics (Latinos) were represented by 8% of photographs of upper lips and 11% of lower lips. Asians (Orientals) were represented by 4% of photographs of upper lips and 5% of lower lips.

TABLE 2
(Demographic Characteristics)

| | Upper Lip | Lower Lip |
|---|---|---|
| Parameter | N = 85 | N = 85 |
| Age (years) | | |
| n | 85 | 85 |
| Mean | 40.2 | 39.5 |
| SD | 15.0 | 14.5 |
| Median | 38.0 | 34.0 |
| Minimum, Maximum | 18, 76 | 18, 75 |
| Age Group N (%) | | |
| 18-34 Years | 40 (47) | 43 (51) |
| 35-54 Years | 28 (33) | 25 (29) |
| >=55 Years | 17 (20) | 17 (20) |
| Gender N (%) | | |
| Male | 32 (38) | 29 (34) |
| Female | 53 (62) | 56 (66) |
| Race/Ethnicity N (%) | | |
| Caucasian | 71 (84) | 68 (80) |
| Hispanic | 7 (8) | 9 (11) |
| African-American | 4 (5) | 4 (5) |
| Asian | 3 (4) | 4 (5) |

### Within-Observer (Intra-Rater) Reliability

Assessments were made by each of the Evaluators at two occasions (Round 1 and Round 2), at least 2 weeks apart. The same set of photographs was used for both rounds, but they were provided to the Evaluators in a different order at each time. The agreement between the ratings of the same observer at the two separate rounds was the indicator of intra-rater reliability. LFS scores for each reviewer for each subject in Round 1 and Round 2 are provided in Listing 1, Appendix 3.

Weighted kappa coefficients for intra-rater reliability were graded according to the following categories:
0-0.19=Poor Agreement
0.20-0.39=Fair Agreement
0.40-0.59=Moderate Agreement
0.60-0.79=Substantial Agreement
0.80-1.0=Almost Perfect Agreement

### Upper Lip

The overall exact agreement was 70% between the Round 1 and Round 2 measurements for the upper lip. The overall within-observer weighted kappa value stratified by rater was 0.81 for the upper lip, indicating almost perfect agreement within raters. The within-observer weighted kappa values varied between 0.70 and 0.87 among the different raters (see Table 3).

TABLE 3
(Intra-Rater Reliability - Upper Lip)

| Agreement | | | | | | |
|---|---|---|---|---|---|---|
| between | | | | | | |
| Round 1 and | All | | | | | |
| Round 2 | Raters | 1 | 2 | 3 | 4 | 5 |
| Upper Lip | | | | | | |
| Exact | 69.9% | 61.2% | 80.0% | 61.2% | 75.3% | 71.8% |
| Agreement | | | | | | |
| Weighted | 0.813 | 0.739 | 0.868 | 0.700 | 0.843 | 0.818 |
| Kappa (95% | (0.781, | (0.657, | (0.809, | (0.609, | (0.781, | (0.749, |
| CI) | 0.844) | 0.820) | 0.927) | 0.790) | 0.906) | 0.888) |

### Lower Lip

The overall exact agreement was 71% between the Round 1 and Round 2 measurements for the lower lip. The overall within-observer weighted kappa value stratified by rater was 0.81 for the lower lip, indicating almost perfect agreement within raters. The within-observer weighted kappa values varied between 0.63 and 0.90 among the different raters (see In-Text Table 4).

TABLE 4
(Intra-Rater Reliability - Lower Lip)

| Agreement | | | | | | |
|---|---|---|---|---|---|---|
| between | | | | | | |
| Round 1 | All | | | | | |
| and Round 2 | Raters | 1 | 2 | 3 | 4 | 5 |
| Lower Lip | | | | | | |
| Exact | 70.6% | 75.3% | 65.9% | 51.8% | 87.1% | 72.9% |
| Agreement | | | | | | |
| Weighted | 0.808 | 0.812 | 0.757 | 0.634 | 0.904 | 0.795 |
| Kappa | (0.776, | (0.737, | (0.679, | (0.541, | (0.847, | (0.713, |
| (95% CI) | 0.841) | 0.887) | 0.835) | 0.727) | 0.960) | 0.876) |

### Between-Observer (Inter-Rater) Reliability

The overall proportion of the observed agreement was calculated for the ten pairs of evaluators, separately for the upper and lower lip scales.

Weighted kappa coefficients for inter-rater reliability were graded according to the following categories:

### 0-0.19=Poor Agreement

### 0.20-0.39=Fair Agreement

### 0.40-0.59=Moderate Agreement

### 0.60-0.79=Substantial Agreement

### 0.80-1.0=Almost Perfect Agreement

Overall unweighted kappa values comparing all of the raters were calculated. Note that these unweighted kappa values do not consider the degree of differences between the ratings, and are therefore generally lower than the weighted kappa values.

### Upper Lip

The exact agreement between the ten pairs of raters varied between 46% and 74% for the upper lip. The between-observer weighted kappa values for the upper lip varied between 0.60 and 0.83, indicating substantial to almost perfect agreement between raters (see In-Text Table 5).

The overall unweighted kappa value comparing all raters simultaneously on the upper lip was 0.47 for Round 1 and 0.50 for Round 2.

### Lower Lip

The exact agreement between the ten pairs of raters varied between 45% and 75% for the lower lip. The between-observer weighted kappa values for the upper lip varied between 0.60 and 0.82, indicating substantial to almost perfect agreement between raters (see Table 6).

The overall unweighted kappa value comparing all raters simultaneously on the lower lip was 0.43 for Round 1 and 0.49 for Round 2.

TABLE 5
(Inter-Rater Reliability - Upper Lip)

| Agreement | | | | | | | | | | |
|---|---|---|---|---|---|---|---|---|---|---|
| between | | | | | | | | | | |
| raters | 1 and 2 | 1 and 3 | 1 and 4 | 1 and 5 | 2 and 3 | 2 and 4 | 2 and 5 | 3 and 4 | 3 and 5 | 4 and 5 |
| Upper lip | | | | | | | | | | |
| Exact | 61.2% | 57.1% | 61.8% | 67.6% | 64.1% | 55.3% | 61.2% | 47.6% | 45.9% | 73.5% |
| Agreement | | | | | | | | | | |
| Weighted | 0.747 | 0.692 | 0.743 | 0.779 | 0.741 | 0.711 | 0.740 | 0.604 | 0.604 | 0.831 |
| Kappa | (0.692, | (0.630, | (0.685, | (0.722, | (0.684, | (0.654, | (0.682, | (0.536, | (0.537, | (0.785, |
| (95% CI) | 0.803) | 0.755) | 0.801) | 0.836) | 0.799) | 0.767) | 0.797) | 0.672) | 0.672) | 0.878) |

TABLE 6
(Inter-Rater Reliability - Lower Lip)

| Agreement | | | | | | | | | | |
|---|---|---|---|---|---|---|---|---|---|---|
| between | | | | | | | | | | |
| raters | 1 and 2 | 1 and 3 | 1 and 4 | 1 and 5 | 2 and 3 | 2 and 4 | 2 and 5 | 3 and 4 | 3 and 5 | 4 and 5 |
| Lower Lip | | | | | | | | | | |
| Exact | 45.3% | 51.8% | 75.3% | 74.7% | 61.8% | 46.5% | 46.5% | 50.6% | 57.1% | 74.1% |
| Agreement | | | | | | | | | | |
| Weighted | 0.607 | 0.625 | 0.815 | 0.802 | 0.725 | 0.616 | 0.620 | 0.629 | 0.680 | 0.807 |
| Kappa (95% | (0.541, | (0.553, | (0.762, | (0.746, | (0.666, | (0.552, | (0.553, | (0.563, | (0.614, | (0.754, |
| CI) | 0.673) | 0.696) | 0.868) | 0.858) | 0.784) | 0.681) | 0.686 | 0.696) | 0.747) | 0.860) |

Results from Live vs. Photographic Validation

For comparison purposes, LFS was evaluated in live subjects as well as photographically. Therefore, a second validation was performed comparing the within-evaluator agreement between the first round of LFS evaluation in live subjects to the second round of validation in photographs of the same subjects.

Assessments were made by each of three Evaluators at two occasions (at least 2 weeks apart) of 39 subjects reflecting the range of the scale ratings 1 to 5 for the upper lip and 39 subjects reflecting the range of the scale ratings 1 to 5 for the lower lip. Intra-rater scores were compared between live and photographic assessment of the same subjects.

### Within-Observer (Intra-Rater) Reliability

The agreement between the ratings of the same observer at the two separate rounds (live vs. photographic) was the indicator of intra-rater reliability. LFS scores for each reviewer for each subject in Round 1 (live assessment) and Round 2 (photo assessment) are provided in Listing 2, Appendix 3.

Weighted kappa coefficients for intra-rater reliability were graded according to the following categories:
0-0.19=Poor Agreement
0.20-0.39=Fair Agreement
0.40-0.59=Moderate Agreement
0.60-0.79=Substantial Agreement
0.80-1.0=Almost Perfect Agreement

### Upper Lip

The overall exact agreement was 60% between the Round 1 (live assessment) and Round 2 (photo assessment) measurements for the upper lip. The overall within-observer weighted kappa value stratified by rater was 0.65 for the upper lip, indicating substantial agreement within raters. The within-observer weighted kappa values varied between 0.62 and 0.68 among the different raters (see Table 7).

TABLE 7
(Intra-Rater Reliability - Upper Lip Live vs. Photo)

| Agreement between | All | | | |
|---|---|---|---|---|
| Round 1 and Round 2 | Raters | 1 | 2 | 3 |
| Upper Lip | | | | |
| Exact | 59.8% | 59.0% | 53.8% | 66.7% |
| Agreement | | | | |
| Weighted | 0.650 | 0.619 | 0.646 | 0.677 |
| Kappa (95% | (0.558, | (0.436, | (0.503, | (0.519, |
| CI) | 0.742) | 0.803) | 0.789) | 0.836) |

### Lower Lip

The overall exact agreement was 52% between the Round 1 (live assessment) and Round 2 (photo assessment) measurements for the lower lip. The overall within-observer weighted kappa value stratified by rater was 0.64 for the lower lip, indicating substantial agreement within raters. The within-observer weighted kappa values varied between 0.61 and 0.68 among the different raters (see Table 8).

TABLE 8
(Intra-Rater Reliability - Lower Lip Live vs. Photo)

| Agreement between | All | | | |
|---|---|---|---|---|
| Round 1 and Round 2 | Raters | 1 | 2 | 3 |
| Lower Lip | | | | |
| Exact | 52.1% | 53.8% | 56.4% | 46.2% |
| Agreement | | | | |
| Weighted | 0.639 | 0.606 | 0.682 | 0.625 |
| Kappa (95% | (0.563, | (0.446, | (0.548, | (0.509, |
| CI) | 0.716) | 0.765) | 0.815) | 0.740) |

### Discussion

The objective of this validation study was to evaluate the 5-graded Lip Fullness Scales (LFS) regarding the within (intra)- and between (inter)-evaluator agreement for the two separate scales, one for the upper lip and one for the lower lip. A total of 85 subjects for the upper lip and 85 subjects for the lower lip were evaluated in Round 1 of the validation. Diverse age groups, genders, and ethnicities were represented in the subjects used to photographically evaluate the LFS in order to evaluate lip fullness in a varied population.

The intra-observer agreement (ability of each evaluator to reproduce their original score at a subsequent time) was evaluated using weighted kappa coefficients interpreted by associated categorical grading. The overall within-observer weighted kappa value stratified by rater was 0.81 for both the upper lip and lower lip, separately. This score indicated almost perfect agreement within the 5 raters for their ability to independently provide an identical score for the same subject during two temporally discrete occasions. The overall exact agreement was consistent for both upper and lower lips (70% and 71%, respectively).

The variation of weighted kappa coefficients for between-observer agreement was consistent between lips, with scores varying from 0.60 to 0.83 (upper) and from 0.60 to 0.82 (lower), indicating substantial to almost perfect agreement between raters for each lip fullness scale.

LFS scoring was compared between live subjects and photographs of the same subjects. The variation of weighted kappa coefficients for intra-observer agreement of overall live vs. photograph was consistent, with overall scores of 0.65 for the upper lip and 0.64 for the lower lip, indicating substantial intra-rater agreement for each lip fullness scale between live and photographic ratings.

Based on the results of intra- and inter-observer ratings using weighted kappa coefficients, it is concluded that the 5-point Lip Fullness Scales (LFS) are considered suitable for use in clinical trials to grade lip fullness.

It should be understood that the foregoing description is only illustrative of the present invention. Various alternatives and modifications can be devised by those skilled in the art without departing from the invention. Accordingly, the present invention is intended to embrace all such alternatives, modifications and variances that fall within the scope of the appended claims.

What is claimed is:

1. A method for measuring the effect of a medical treatment on the size of lips, comprising:
(a) providing a scale of at least four reference images exhibiting varying lip sizes and assigning a unique indicator to each of the at least four reference images;
(b) examining a lip of a human subject to be augmented and selecting one from among of at least four different reference images most closely corresponding in lip size and identifying the corresponding unique indicator;
(c) introducing into the lip of the human subject a filler or an implant to augment the size of the lip;
(d) examining the lip after introduction of the filler or the implant and selecting one of the at least four different reference images most closely corresponding in lip size and identifying the corresponding unique indicator; and (e) comparing the unique indicator of the lip before introduction of the filler or the implant and the unique indicator of the lip after introduction of the filler or the implant to determine if they are different.

2. The method of claim 1, wherein there are four to six reference images.

3. The method of claim 1, wherein there are five reference images.

4. The method of claim 3, wherein the five reference images collectively have unique indicators ranging from numerals 1 to 5.

5. The method of claim 1, wherein the unique indicators assigned to the at least four reference images are numerals.

6. The method of claim 1, wherein lip size is selected from the group consisting of upper lip volume, lower lip volume, and total lip volume.

7. The method of claim 1, wherein the variation in lip size relates to a dimension selected from the group consisting of total vermilion height, upper red lip median height, upper red lip lateral height, and lower lip median height, upper lip vermilion area, lower lip vermilion area, and combinations of the foregoing.

8. The method of claim 1, wherein examination of the lip before and after introduction of the filler or the implant is carried out by computer, and wherein selection of one from among the at least four different reference images before and after introduction of the filler or the implant is carried out by computer.

9. The method of claim 1, wherein examination of the lip before and after introduction of the filler or the implant is carried out visually, and wherein selection of one from among the at least four different reference images before and after introduction of the filler or the implant is carried out by computer.

10. A method for counseling a human subject undertaking augmentation of lips, comprising:
(a) visually examining a lip of the human subject and comparing it to a scale of at least four reference images exhibiting human lips of varying sizes; (b) selecting a first reference image from among the at least four different reference images that corresponds most closely in lip size to that of the human subject wherein the first reference image does not exhibit the largest lip size among the at least four different reference images; (c) selecting a second reference image from among the at least four different reference images that exhibits lips of larger size than that of the first reference image; and (d) allowing the human subject to visually compare the lip size exhibited in the first reference image with the lip size exhibited in the second reference image.

11. The method of claim 10, wherein the second image is next size larger than the first image.

12. The method of claim 10, wherein the second image is two or more sizes larger than the first image.

13. The method of claim 10, further comprising allowing the human subject to communicate how full he or she wants his or her lips to be after visually comparing the first reference image and the second reference image.

14. A method for developing a scale for measuring differences in lip size in human subjects, comprising:
(a) developing a scale of at least four visual reference images exhibiting varying lip sizes and having unique indicators assigned thereto;
(b) subjecting the scale to a panel test of a plural number of human subjects and a plural number of evaluators who each visually examine a lip of each of the plural number of human subjects and assign a unique indicator to each lip; and
(c) approving the scale as viable for use in human subjects if the weighted kappa coefficient for each of the unique indicators is from 0.40 to 1.0 with an associated 95% confidence interval.

15. The method of claim 14, wherein the weighted kappa coefficient is from about 0.60 to 1.0.

16. The method of claim 14, wherein the weighted kappa coefficient is from about 0.80 to 1.0.

17. A method for determining the amount of filler or implant needed to augment the lips of a human subject, comprising:
(a) examining a lip of the human subject and comparing it to a scale of at least four reference images exhibiting human lips of varying sizes;
(b) selecting a first reference image from among the at least four different reference images that corresponds most closely in lip size to that of the human subject wherein the first reference
image does not exhibit the largest lip size among the at least four different reference images;
(c) selecting a second reference image from among the at least four different reference images that exhibits lips of larger size than that of the first reference image and that substantially corresponds to an augmented lip size desired by the human subject; and
(d) ascertaining the amount of filler or implant needed on the basis of a predetermined relative amount relationship between the first reference image and the second reference image.

18. The method of claim 17, wherein the examining of the lip, the selecting of the first reference image, and the selecting of the second reference image are carried out by computer.

19. The method of claim 17, wherein the examining of the lip, the selecting of the first reference image, and the selecting of the second reference image are carried out visually.
